(19) United States
(12) Patent Application Publication
(10) Pub. No.: US 2025/0051588 A1
(43) Pub. Date: Feb. 13, 2025

(54) SUBSTRATE COATED WITH A POLYMER OBTAINED BY GRAFTING AN AQUEOUS GRAFTING SOLUTION

(71) Applicant: ACTIV' BIOMAT, Roissy Aeroport CDG (FR)

(72) Inventors: Daniel BLANQUAERT, Paris (FR); Véronique MIGONNEY, Eaubonne (FR)

(73) Assignee: ACTIV' BIOMAT, Roissy Aeroport CDG (FR)

(21) Appl. No.: 18/722,685

(22) PCT Filed: Dec. 20, 2022
(86) PCT No.: PCT/FR2022/052443

§ 371 Date: Jul. 3, 2024

(30) Foreign Application Priority Data
Dec. 23, 2021 (FR) FR2114392

(51) Int. Cl. C09D 5/44; C09D 151/00

(52) U.S. Cl. CPC C09D 5/4476; C09D 151/003

(57) ABSTRACT

A substrate coated with a polymer obtained by grafting an aqueous monomer grafting solution comprising a mixture that includes sodium styrene sulphonate (NaSS) and methacrylic acid (MA) or acrylic acid (AA). The mixture has 10 to 90 mol % of sodium styrene sulphonate and 10 to 90 mol % of methacrylic acid or acrylic acid. The substrate is selected from among polyesters, vinyl polymers, polyacrylics and polymethacrylics, PEEK, silicones, natural polymers, natural or artificial celluloses, collagens, glycopolymers, ceramics, metals and metal alloys, and in particular Ti and alloys thereof and Ni—Ti alloys. The aqueous grafting solution has, in addition to the mixture which represents 90 to 99 mol % of the aqueous grafting solution, 1 to 10 mol % of hydroxyethylmethacrylate (HEMA).

The present invention relates to a substrate coated with a polymer obtained by grafting an aqueous grafting solution comprising monomers, with the aim of obtaining a polymer grafted on a substrate. The invention also relates to a grafting method.

Cell adhesion or adherence is an essential function of the organism which occurs naturally during the formation of tissues or organs. Cell adhesion can occur between cells, and intercell adhesion is thus referred to, that is between cells and a substrate. Cell/substrate adhesion occurs as soon as a cell is in contact with a natural surface, such as a membrane, or also the extracellular or synthetic matrix, such as a medical device.

Cell adhesion can occur under in vitro laboratory conditions or in vivo conditions, during the implantation of a medical device. This step is determinant for further interactions between cells and the surface of a medical device.

Cell proliferation is a natural phenomenon during which cells multiply to form tissues; it follows cell adhesion.

The chemical, physico-chemical or physical nature of the surface of the substrate very highly modifies the interactions which develop between the cells and the substrate during cell adhesion and proliferation. With the aim of modulating cell adhesion and proliferation, different surface treatments have been developed; among these, there are treatments for decreasing, even inhibiting cell adhesion and proliferation.

In the prior art, techniques for covering or coating substrates with highly hydrophilic, natural or synthetic molecules or macromolecules or by hydrogels are known, making it possible to prevent interactions between the cells and these substrates, and consequently, decrease cell adhesion and proliferation. Also, the modification of substrates is known by coating or fixing of cytotoxic agents, such as quaternary ammoniums or silver salts to prevent any adhesion and consequently, any cell proliferation. Simpler, “mechanical”-type methods have also been proposed to create a physical barrier to prevent the cells from reaching a surface.

These techniques of the prior art are not satisfactory for the following reasons: the coating by highly hydrophilic molecules or by hydrogels can make it possible to decrease the adhesion of cells on a substrate, however the coating is rarely obtained by a covalent grafting, and it is weakened and its integrity is difficult to control during drying, sterilising and more simply, preserving and storing steps.

Another major problem of grafted coatings resides in the grafting rate, which is often insufficient.

The present invention aims to overcome the abovementioned disadvantages of the prior art, by defining an aqueous grafting solution, associating several monomers, capable of generating a polymer grafted on a substrate, with a highly improved grafting rate.

The aqueous grafting solution comprises the following monomers:
sodium styrene sulphonate (NaSS),
methacrylic acid (MA) or acrylic acid (AA), and
hydroxyethylmethacrylate (HEMA).

Sodium styrene sulphonate (NaSS) has properties making it possible to provide a glycosaminoglycan-like character and a controlled biological response of the modified surface.

Methacrylic acid (MA) or acrylic acid (AA) has properties making it possible, when it is combined with Nass, to decrease cell adhesion and proliferation. It also contributes to providing the glycosaminoglycan-like character on the surface.

Regarding hydroxyethylmethacrylate (HEMA), it has hydrophilic properties which improve the wettability of the substrate, and makes it possible, according to the quantity of “hydroxyl” groups introduced, to modulate the inflammatory response.

According to the invention, a substrate is obtained, which is coated with a polymer obtained by grafting an aqueous monomer grafting solution comprising a mixture that comprises:
sodium styrene sulphonate (NaSS), and
methacrylic acid (MA) or acrylic acid (AA), and
this mixture comprising 10 to 90 mol % of sodium styrene sulphonate (NaSS) and 10 to 90 mol % of methacrylic acid (MA) or acrylic acid (AA),
wherein the substrate is selected from among polyesters, vinyl polymers, polyacrylics and polymethacrylics, PEEK, silicones, natural polymers, natural or artificial celluloses, collagens, glycopolymers, ceramics, metals and metal alloys, and in particular Ti and alloys thereof and Ni—Ti alloys, and
the aqueous grafting solution comprises, in addition to said mixture which represents 90 to 99 mol % of the aqueous grafting solution, to 1 to 10 mol % of hydroxyethylmethacrylate (HEMA).

The percentages of NaSS and of MA or AA in the mixture and the percentages of mixture and of HEMA in the grafting solution must be distinguished.

In addition to a very high grafting rate with HEMA between 1 and 10 mol %, the grafted polymer is a copolymer capable of minimising, even of inhibiting the cell adhesion eukaryotic like of cells, for example osteoblast, fibroblast, keratinocyte, endothelial, epithelial cells on the substrate, and of minimising or inhibiting the proliferation of adherent cells on this same substrate.

Advantageously, the aqueous grafting solution comprises 45 to 49.5 mol % of sodium styrene sulphonate (NaSS), 45 to 49.5 mol % of methacrylic acid (MA) or acrylic acid (AA), and 1 to 10 mol % of hydroxyethylmethacrylate (HEMA).

According to a first embodiment, the aqueous grafting solution can comprise 45% mol % of sodium styrene sulphonate (NaSS), 45 mol % of methacrylic acid (MA) or acrylic acid (AA) and 10 mol % of hydroxyethylmethacrylate (HEMA).

According to a second embodiment, the aqueous grafting solution can comprise 49.5 mol % of sodium styrene sulphonate (NaSS), 49.5 mol % of methacrylic acid (MA) or acrylic acid (AA), and 1 mol % of hydroxyethylmethacrylate (HEMA).

According to a third embodiment, the aqueous grafting solution can comprise 47.5 mol % of sodium styrene sulphonate (NaSS), 47.5 mol % of methacrylic acid (MA) or acrylic acid (AA), and 5 mol % of hydroxyethylmethacrylate (HEMA).

A protection for such aqueous grafting solutions could be sought.

The invention also defines a grafting method to apply, on a substrate, a polymer obtained by grafting the aqueous grafting solution such as defined above on a substrate such as defined above, the grafting being a radical grafting or an electrografting.

The grafting step, strictly speaking, is preferably preceded by a surface activation step. On titanium, for example, the grafting method can be of the radical type with a surface activation step carried out by anodic oxidation, such as described in document WO2017068272. This prior activation step is followed by thermal polymerisation or by UV polymerisation. For the other types of substrate, a surface activation step is also recommended, otherwise it will be difficult to polymerise from the surface.

The spirit of the invention resides in the fact of selecting statistic copolymerisation of three-monomers carrying chemical groups, defined and selected for their specific feature and for the properties that they will provide to the surface. In other words, the copolymerisation of NaSS, of MA and of HEMA in the proportions defined makes it possible to increase the grafting rate, but also to control cell adhesion and proliferation on these surfaces, while maintaining a control of the inflammatory response.

Thus, the grafting of a NaSS, MA and HEMA copolymer on titanium or a titanium alloy makes it possible to inhibit the adhesion of osteoblast and fibroblast cells, and to prevent the adhesion of cells and tissues on osteosynthetic plates intended to be explanted.

Measurements of the grafting rate have been taken in the laboratory under precise and controlled conditions on different Ti alloy pellets. Ti pellets would also have been able to be used.

First, a 50 mol %/50 mol % NaSS and MA mixture (without HEMA) has been grafted on a basic pellet P0. A basic grafting rate T0 of around 5 micrograms/cm² has thus been measured.

Then, three other pellets P1, P2 and P3 have been coated by grafting with a NaSS, MA and HEMA mixture of the invention:
Pellet 1: grafted with a solution containing 49.5 mol % of NaSS, 49.5 mol % of MA and 1 mol % of HEMA, with a measured grafting rate T1 of around 10 micrograms/cm²,
Pellet 2: grafted with a solution containing 47.5 mol % of NaSS, 47.5 mol % of MA and 5 mol % of HEMA, with a measured grafting rate T2 of around 15 micrograms/cm²,
Pellet 3: grafted with a solution containing 45 mol % of NaSS, 45 mol % of MA and 10 mol % of HEMA, with a measured grafting rate T3 of around 20 micrograms/cm².

It has thus been able to be observed that T1 is around 2 times greater than T0, that T2 is around 3 times greater than T0, and that T3 is around 4 times greater than T0. T1 is half of T3 for a percentage 10 times lower.

Therefore, it can be concluded that a very low HEMA percentage, namely 1%, is sufficient for significantly increasing (100%) the grafting rate of an NaSS and MA mixture. And with an even lower percentage of 10%, the grafting rate is increased, but less rapidly.

This amplified grafting rate naturally has a significant impact on the inhibition of cell adhesion and proliferation.

It must be noted that MA (methacrylic acid) can be replaced by AA (acrylic acid) in the NaSS and HEMA mixture, with equivalent results.

1. A substrate coated with a polymer obtained by grafting an aqueous monomer grafting solution comprising a mixture that comprises:
sodium styrene sulphonate, and
methacrylic acid or acrylic acid, and
this mixture comprising 10 to 90 mol % of sodium styrene sulphonate and 10 to 90 mol % of methacrylic acid or acrylic acid,
wherein the substrate is selected from among polyesters, vinyl polymers, polyacrylics and polymethacrylics, PEEK, silicones, natural polymers, natural or artificial celluloses, collagens, glycopolymers, ceramics, metals and metal alloys, and in particular Ti and alloys thereof and Ni—Ti alloys, and
the aqueous grafting solution comprises, in addition to said mixture which represents 90 to 99 mol % of the aqueous grafting solution, 1 to 10 mol % of hydroxyethylmethacrylate.

2. The substrate according to claim 1, wherein the aqueous grafting solution comprises 45 to 49.5 mol % of sodium styrene sulphonate, 45 to 49.5 mol % of methacrylic acid or acrylic acid, and 1 to 10 mol % of hydroxyethylmethacrylate.

3. The substrate according to claim 1, wherein the aqueous grafting solution can comprise 45% mol % of sodium styrene sulphonate, 45 mol % of methacrylic acid or acrylic acid and 10 mol % of hydroxyethylmethacrylate.

4. The substrate according to claim 1, wherein the aqueous grafting solution comprises 49.5 mol % of sodium styrene sulphonate, 49.5 mol % of methacrylic acid or acrylic acid, and 1 mol % of hydroxyethylmethacrylate.

5. The substrate according to claim 1, wherein the aqueous grafting solution comprises 47.5% mol % of sodium styrene sulphonate, 47.5 mol % of methacrylic acid or acrylic acid and 5 mol % of hydroxyethylmethacrylate.

6. A grafting method to apply, on a substrate, a polymer obtained by grafting an aqueous grafting solution, comprising a monomer mixture that comprises:
sodium styrene sulphonate, and
methacrylic acid or acrylic acid,
this monomer mixture comprising 10 to 90 mol % of sodium styrene sulphonate and 10 to 90 mol % of methacrylic acid or acrylic acid,
wherein the aqueous grafting solution comprises, in addition to said monomer mixture which represents 90 to 99 mol % of the aqueous grafting solution, 1 to 10 mol % of hydroxyethylmethacrylate,
wherein the grafting is a radical grafting.

7. The grafting method to apply, on a substrate, a polymer obtained by grafting an aqueous grafting solution, comprising a monomer mixture that comprises:
sodium styrene sulphonate, and
methacrylic acid or acrylic acid,
this monomer mixture comprising 10 to 90 mol % of sodium styrene sulphonate and 10 to 90 mol % of methacrylic acid or acrylic acid,
wherein the aqueous grafting solution comprises, in addition to said monomer mixture which represents 90 to 99 mol % of the aqueous grafting solution, 1 to 10 mol % of hydroxyethylmethacrylate,
wherein the grafting is an electrografting.

8. The grafting method according to claim 6, wherein the aqueous grafting solution comprises 45 to 49.5 mol % of sodium styrene sulphonate, 45 to 49.5 mol % of methacrylic acid or acrylic acid, and 1 to 10 mol % of hydroxyethylmethacrylate.

9. The grafting method according to claim 6, wherein the aqueous grafting solution comprises 45% mol % of sodium styrene sulphonate, 45mol % of methacrylic acid or acrylic acid and 10 mol % of hydroxyethylmethacrylate.

10. The grafting method according to claim 6, wherein the aqueous grafting solution comprises 49.5 mol % of sodium styrene sulphonate, 49.5 mol % of methacrylic acid or acrylic acid, and 1 mol % of hydroxyethylmethacrylate.

11. The grafting method according to claim 6, wherein the aqueous grafting solution comprises 47.5 mol % of sodium styrene sulphonate, 47.5 mol % of methacrylic acid or acrylic acid and 5 mol % of hydroxyethylmethacrylate.
